(19) United States
(12) Patent Application Publication
(10) Pub. No.: US 2004/0225178 A1
(43) Pub. Date: Nov. 11, 2004

(54) Apparatus and methods for treating symptoms of disease and conditions of the ear

(76) Inventor: Timothy J. Kriewall, Jacksonville, FL (US)

Correspondence Address: Epstein, Edell, Shapiro, Finnan & Lytle, LLC, Suite 400, 1901 Research Boulevard, Rockville, MD, 20850, US

(21) Appl. No.: 10/151,691

(22) Filed: May 21, 2002

(51) Int. Cl. H04R 25/00; A61N 1/00

(52) U.S. Cl. 600/25

(57) ABSTRACT

A pressure waveform is generated and applied to a middle ear space of a human ear to treat symptoms of Meniere's disease or endolymphatic hydrops. The waveform includes a varying pressure waveform combined with a static pressure and has a variable first derivative that can be approximated by a plurality of sinusoidal waves combined as in a Fourier series.

### BACKGROUND OF THE INVENTION

1. Field of the Invention

The present invention relates to apparatus and methods for influencing the fluid system of the inner ear and, more particularly, to such apparatus and methods for treating symptoms of diseases and conditions of the ear, such as Meniere's disease and endolymphatic hydrops.

2. Description of the Related Art

Meniere's disease is a chronic disease from which millions of people suffer. The origin of Meniere's disease is believed to be an imbalance in the hydrodynamic system of the inner ear, described as endolymphatic hydrops. In addition to the severity of the symptoms of Meniere's disease, which include fluctuating hearing loss, fluctuating tinnitus, fluctuating sense of fullness in the ear and fluctuating vertigo, the unpredictable onset of the symptoms creates a major handicap for sufferers of Meniere's disease.

As illustrated in FIG. 1, the human ear 10 includes three primary spaces, the outer ear canal 12, also known as the external acoustic meatus, the middle ear 14, and the inner ear 16. The middle ear, also known as the tympanic cavity, is adjacent the outer ear canal and is separated from the outer ear canal by the tympanic membrane 18, also known as the ear drum. The inner ear includes the cochlea 20 formed of the scala vestibuli 22 and the scala tympani 24 which surround the cochlear duct 26 (cross-hatched to simplify visualization). The cochlear duct is filled with endolymphatic fluid supplied by the endolymphatic sac 28. The scala vestibuli 22 and scala tympani 24 are filled with perilymph fluid that moves in response to displacement of the footplate or base 29 of the stapes 30 in the oval window 32. The stapes is one of a series of small bones (ossicles) in the middle ear connecting the tympanic membrane 18 with the inner ear. The inner ear is separated from the middle ear by a partition formed of the stapes footplate at the oval window and the round window membrane 34. Movement of the stapes footplate in the oval window 32 causes the perilymph fluid to move within the scala vestibuli affecting the endolymphatic fluid within the cochlear duct to effect hearing. The round window membrane 34 separates the scala tympani 24 of the inner ear from the middle ear 14 and operates to dissipate waves formed in the perilymph fluid.

The symptoms of Meniere's disease are believed to be caused by endolymphatic hydrops, an excessive buildup of endolymphatic fluid in the cochlea. Meniere's disease is typically characterized by varying degrees of four classic symptoms: 1) fluctuating hearing loss, the extent of which increases over time; 2) fluctuating tinnitus, causing various sounds, described as whining, roaring or other sounds; 3) fluctuating sense of fullness, or a “plugged ear” sensation similar to a sensation one experiences upon descending from a mountain and being unable to clear or equalize the pressure in one's ear; and 4) fluctuating vertigo, or dizziness that can range from mild to severe. As used herein, the terms “symptoms of Meniere's disease” means some or all of the above symptoms in that the method and apparatus of the present invention can provide treatment for any of the above symptoms, individually or together, which are caused by endolymphatic hydrops.

An early method of treating a person with Meniere's disease was developed empirically and includes placing the patient in a pressure chamber to alleviate the symptoms. The theory of the treatment is to place pressure on the inner ear fluids to attempt to reduce the amount of fluid in the endolymph system, specifically the endolymph fluid within the scala media (not shown) of the cochlear duct 26.

Another method includes applying air pressure pulses to the middle ear by way of a hole through the tympanic membrane. The hole allows the pressure pulses to pass from the outer ear canal into the middle ear. It is believed that the round window membrane moves in response to the pressure changes and transfers the movement/pressure to the perilymph fluid, which resultantly transfers the motion/pressure to the endolymph fluid through membranes separating those two distinct fluids. The oval window may also act to transfer varying pressure to the perilymph fluid; and, accordingly, hereinafter references to displacement of the round window membrane also are meant to include displacement of the stapes footplate at the oval window. U.S. Pat. No. 4,757,807 and No. 4,757,807, WO Publications No. 83/02556, No. 93/08775, No. 97/23178, No. 00/01331, No. 00/01346 and No. 00/10484, European Patent No. 266474 B 1, Acta Otolaryngol 102:186-193,1986, Acta Otolaryngol 91:55-64, 1981, Acta Otolaryngol 102:403-409, 1986, Laryngoscope V.92, No. 11, 1982; 1285-92, Carlborg et al 1982 V. 91, No. 2, American Journal of Otology 18:726-733, Acta Otolaryngol 2000 543:99-101 and Acta Otolaryngol 2001 121:616-621 are representative of the above method which is also exemplified by the Meniett portable pressure pulse generator sold by Medtronic Xomed, Inc., Jacksonville, Fla.

In order to practice the above method, a hole formed in the tympanic membrane 18 is fitted with a ventilation tube 36, as shown in FIG. 1. After the ventilation tube 36 is inserted into the tympanic membrane, pressure pulses are generated and transmitted into the outer ear canal 12 by way of a tube having an ear plug sealing the tube against the walls of the outer ear canal so that the pressure pulses vary the pressure in the middle ear through the ventilation tube to influence the fluid system of the inner ear through the round window membrane. That is, the ventilation tube 36 allows air to pass from the outer ear canal 12 to the middle ear 14, thereby allowing the pressure pulses applied to the outer ear canal to cause the pressure in the middle ear to fluctuate. Although the actual mechanisms are still not fully understood, one theory of action that reduces endolymphatic hydrops is that the action of the pressure pulses on the fluid system combine with other physiologic reactions in the ear to force excess endolymph fluid into the endolymphatic sac 28.

The above method of treating Meniere's disease relies on applying a series of pressure pulses biased by a positive pressure P_{S}, as illustrated in FIG. 2. The pressure pulses are applied to the outer ear canal to transmit varying pressure changes through the ventilation tube 36 into the middle ear in order to displace the round window membrane 34. The series of pressure pulses developed as a result of empirical observations have been used to treat patients suffering from Meniere's disease.

The pressure pulses applied in the past have included sine waves, static and alternating components, a predetermined overpressure superimposed on pressure oscillations, square waves and low frequency sine waves superimposed on square wave pressure pulses. The Densert et al U.S. Pat. No. 6,159,171 shows pressure pulses generated by the Meniett portable pressure pulse generator in FIG. 3 thereof and provides a description thereof in columns 3 and 4.

### SUMMARY OF THE INVENTION

In view of the above, it is a primary object of the present invention to utilize pressure pulse waveforms for treating the symptoms of Meniere's disease or endolymphatic hydrops defined in analytical or theoretical terms as opposed to empirical terms.

A further object of the present invention is to describe a pressure waveform applied to the middle ear as a mathematical function in order to measure therapeutic benefit as a function of the simple variables forming the pressure waveform.

It is another object of the present invention to define attributes of pressure pulse waveforms for treating the symptoms of Meniere's disease or endolymphatic hydrops in a theoretical manner to permit waveforms having the defined attributes to be generated.

A further object of the present invention is to treat the symptoms of Meniere's disease or endolymphatic hydrops by applying a pressure pulse waveform having the attributes of a constant pressure with a superimposed variable pressure where the first derivative of the pulses of the pressure waveform is not equal to zero and is variable thereby permitting the use of pressure waveforms of varying shapes and attributes or components.

Another object of the present invention is to generate a variable pressure waveform having a theoretically determined pattern suitable for treating patients having different physical characteristics, such as different anthropometrical sizes, or individual symptoms of the type associated with Meniere's disease and endolymphatic hydrops.

Yet another object of the present invention is to treat a patient who suffers from an ear disease with a pressure wave that varies in pressure in a pattern determined to be best suited to the patient's condition.

Still another object of the present invention is to provide methods and apparatus for use in researching treatment of symptoms of ear diseases, such as Meniere's disease.

Another object of the present invention is to provide a theoretical basis for varying pressure waveforms that is independent of the means for delivering the waveforms.

The aforesaid objects are achieved individually and in combination, and it is not intended that the invention be construed as requiring two or more of the objects to be combined unless expressly required by the claims attached hereto.

In accordance with an aspect of the present invention, a pressure waveform is applied to the middle ear of the human ear having a first derivative that varies and shape other than that of a single frequency sine wave. According to another aspect of the present invention, a pressure waveform is applied to the middle ear of the human ear that can be approximated by a combination of a plurality of sine waves having different frequencies. Such pressure waveforms can be generated by devices to treat Meniere's disease or other individual ear ailments or conditions such as tinnitus, vertigo, hearing loss and fullness of the ear, often associated with Meniere's disease and endolymphatic hydrops.

The above and still further objects, features and advantages of the invention will become apparent upon consideration of the following description and descriptive figures of specific embodiments thereof. While the description goes into specific details of the present invention, it should be understood that variations would be apparent to those skilled in the art based on the descriptions herein.

### BRIEF DESCRIPTION OF THE DRAWINGS

FIG. 1 is a broken section of a human ear showing a portion of the outer ear, middle ear and inner ear.

FIG. 2 is a graph of a pressure wave in the form of a sine wave biased by a positive pressure in accordance with the prior art.

FIG. 3 is a broken section of a human ear and a block diagram of a treatment device for use in displacing the round window membrane in response to a composite pressure wave according to the present invention.

FIGS. 4A and 4B are graphs of a square wave pressure waveform and its first derivative, respectively, for use in accordance with the present invention.

FIGS. 5A and 5B are graphs of a triangular sawtooth wave and its first derivative, respectively, for use in accordance with the present invention.

FIGS. 6A, 6B and 6C are graphs showing a first sine wave having a first frequency (FIG. 6A) combined with a second sine wave having a second frequency different from the first frequency (FIG. 6B) to produce the waveform of FIG. 6C.

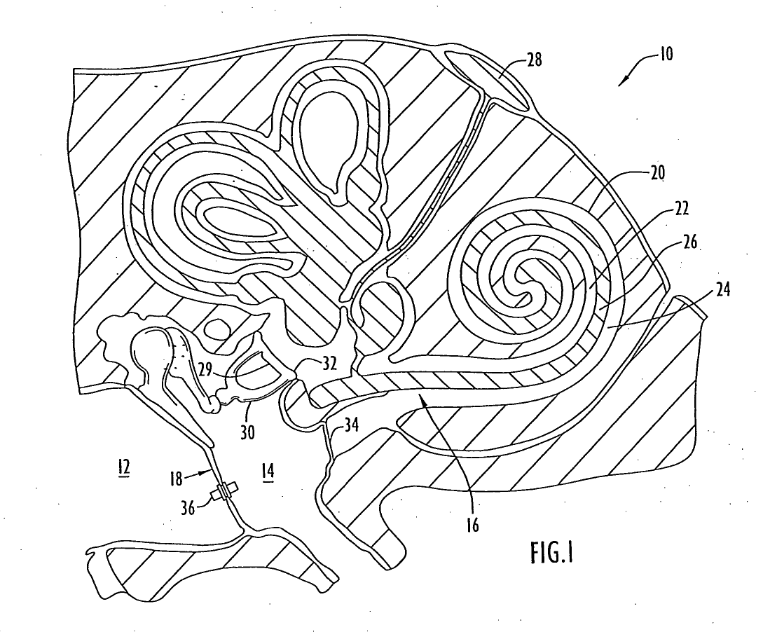

### DETAILED DESCRIPTION

The embodiments described below are described with reference to the above drawings, in which like reference numerals designate like components.

In accordance with the present invention, it has been found that a pressure waveform having a varying first derivative can be used to treat the symptoms of Meniere's disease and endolymphatic hydrops and to provide a framework in which researchers can experiment and analyze various types of waveforms that are effective in treating Meniere's disease. Pressure waveforms that effectively treat the symptoms of Meniere's disease are described using mathematical functions. Knowing those mathematical functions allows for a theoretical approach to developing effective therapeutic treatments. More particularly, the therapeutic benefits of various treatments can be measured as a function of the variables that describe such mathematical functions. For example, the sinusoidal waveform used in conventional treatment methods is described below in equation (1).

P_{DC}+Pₒ sin(ωt) (1)

The variable P_{DC} is the magnitude of a constant pressure, Pₒ is the amplitude of the variable pressure, and ω is the frequency of oscillation of the sinusoidal waveform. t is the time from the onset of the waveform.

Equation 1 describes the waveform used in the conventional method described above relating to the Meniett device. It has been found, however, that all waveforms that have a variable first derivative can be effective in treating the symptoms of Meniere's disease if the attributes of the waveforms are approximately the same. For example, if the amplitude and frequency of a varying waveform are approximately the same as those used in the conventional methods, then the resulting therapeutic benefit for a given patient is expected to be similar. Improvements in the therapeutic benefits of pressure treatments can be gained by modifying the descriptive attributes of the pressure waveform or the shape of the waveform. A mathematical description of a pressure waveform used to treat the symptoms of Meniere's disease will enable researchers to categorize the waveform attributes that may, in fact, optimize the therapy.

Referring to FIG. 3, a pressure waveform for use in treating symptoms of Meniere's disease in accordance with the present invention is generated by device 40 including a pressure generator 42 that generates a pneumatic pressure waveform at the output or outlet of the device 40. The pressure generator 42 is controlled by a control unit 44 to generate the waveform specified by a signal from the control unit. For example, the control unit can include a microprocessor and software instructions that output control signals to the pressure generator to vary certain aspects of the pressure waveform. The control unit is programmed to control the pressure generator to generate the pressure waveform with specific characteristics.

Both the control unit and the pressure generator are powered by a power supply 46 that supplies energy to create the pressure waveform. The power supply can be electrical and can receive power from an AC power source or, alternatively, from a DC power source such as from a sufficiently charged battery suitable for driving not only the control unit but also the pressure generator to generate pneumatic pressure waveforms. The power source may also be pneumatic or manual.

The pneumatic pressure waveform output from device 40 is delivered to the outer ear canal 14 by way of a delivery tube 48 in communication with the pressure generator at one end and at the other end with an ear plug 50. The ear plug 50 has an aperture 52 that is formed at the end of a channel through the ear plug, and the pressure wave generated by the treatment device 40 is, thus, communicated to outer ear canal 14. The ear plug 50 is shaped so as to form a seal with the walls of the outer ear canal when the patient places the ear plug in his or her ear. Accordingly, pressure waves emanating from aperture 52 are communicated to the middle ear 14 via the ventilation tube 36. In this manner pressure changes resulting from the pressure waveform are transmitted to the inner ear by displacing portions of the partition between the middle ear and the inner ear, the partition including the round window membrane 34 and the stapes footplate at oval window 32 thereby treating the symptoms of Meniere's disease.

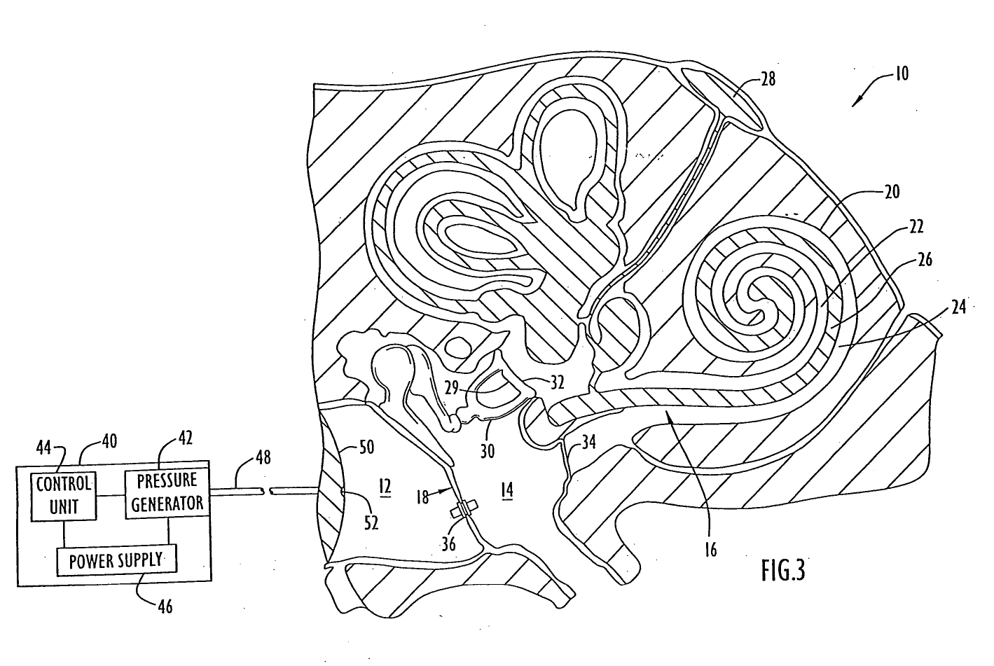

The control unit 44 controls the pressure generator to produce a variety of different shaped pressure waveforms. Examples of such waveforms are shown in FIGS. 4A, 4B, 5A and 5B. The waveform in FIG. 4A is a square wave that is superimposed on a constant static pressure P_{S}. The square wave varies between the static pressure level P_{S} and a peak pressure P_{P}. When the parameters of the square wave are adjusted appropriately, the first derivative of the square wave, shown in FIG. 4B, has a varying waveform which decays exponentially until the waveform transitions from P_{P} to P_{S}, after which the first derivative increases in an exponential manner until the next pressure pulse occurs and the pattern repeats itself. Thus, by theoretically (non-empirically) selecting certain parameters of the pressure wave, such as the duty cycle and length of pulses, a varying first derivative is produced. Accordingly, control unit 44 can be programmed to drive the pressure generator to produce a square wave pressure waveform as illustrated in FIG. 4 for use in treating the symptoms of Meniere's disease. It has been found that the endolymphatic sac 28 will respond according to the first derivative of the pressure waveform applied to the outer ear canal and, hence, the middle ear space in that displacement or movement of the partition will force endolymphatic fluid back into the endolymphatic sac and thereby reduce the symptoms of Meniere's disease. In other words, for there to be a therapeutic benefit, a waveform must have a first derivative that is not equal to zero some of the time.

The pressure waveform in FIG. 5A is a triangular sawtooth waveform superimposed on a static pressure P_{S}. The triangular sawtooth pressure waveform varies from P_{S} to a peak pressure P_{P} with the pressure increasing linearly from P_{S} to P_{P} prior to repeating itself. The first derivative of the triangular sawtooth waveform is shown in FIG. 5B and produces variations at the points transitioning from pressure level P_{S} to pressure level P_{P}. These changes in the first derivative of the pressure waveform force the endolymphatic fluid back into the endolymphatic sac 28 and thereby ameliorate the symptoms of Meniere's disease.

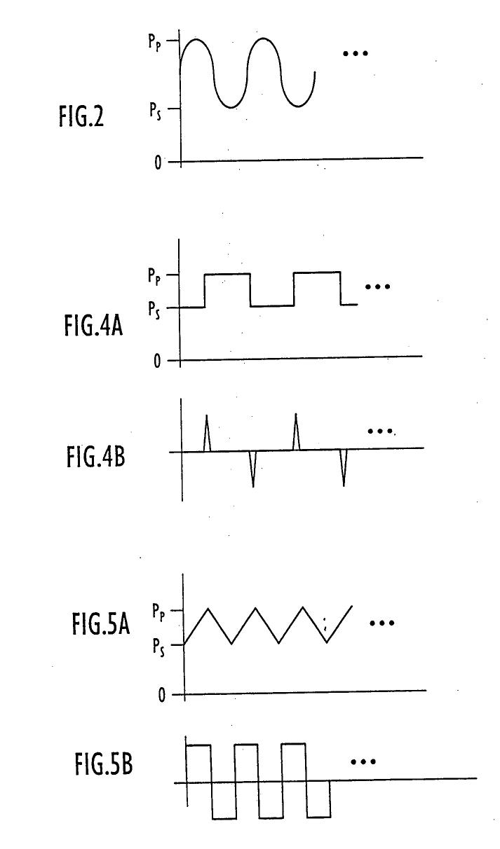

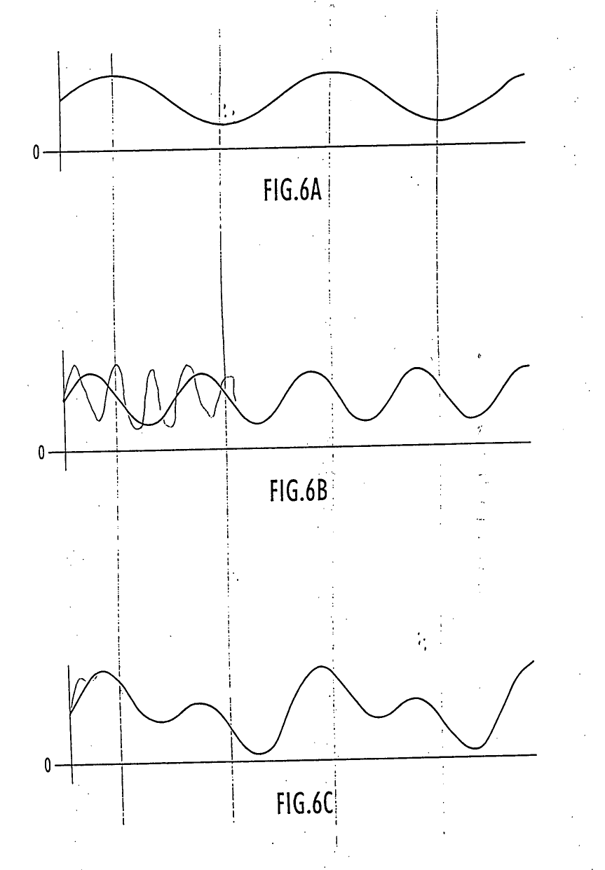

The treatment device and method according to the present invention utilizes a composite waveform produced from a plurality of fundamental waveforms, in which the composite waveform has a varying first derivative. For example, the control unit 44 can be programmed to drive the pressure generator 42 to produce a waveform that can be approximated by the combination of a plurality of sinusoidal waveforms at different frequencies as shown in FIGS. 6A through 6B. A first sinusoidal wave, having a first frequency as shown in FIG. 6A, is combined with a second sinusoidal signal having a higher second frequency as shown in FIG. 6B. The two waveforms shown in FIGS. 6A and 6B are combined to produce a composite waveform illustrated in FIG. 6C where the pressure varies in a repeating, but irregular pattern, as compared to the single sinusoidal waveform shown in FIG. 2. The waveforms shown in FIGS. 6A through 6B are biased by a constant pressure. Accordingly, the waveform shown in FIG. 6C is a pressure waveform with a constant pressure combined with a variable pressure where the first derivative of the pressure waveform is not equal to zero and is itself variable. Although only two waveforms are shown in FIGS. 6A and 6B for purposes of explanation, a third or even more waveforms can be combined to produce the composite pressure waveform.

The waveform shown in FIG. 6C can be described in terms of a Fourier series. Accordingly, the pressure waveform generated by the treatment device 40 can be described as a Fourier series and/or as a waveform that is variable and has a variable first derivative. By programming the control unit to control the pressure generator 42 to produce a waveform corresponding to a plurality of sinusoidal waveforms, the treatment device 40 can produce many different varying pressure waveforms with varying first derivatives that can be used to treat the symptoms of Meniere's disease.

The pressure waveform produced by the treatment device 40 can be approximated by a combination of a plurality of sinusoidal waves having different frequencies. Thus, the pressure waveform output by device 40 is a waveform approximated by a Fourier series. A Fourier series is described mathematically below in equation (2), with the Fourier coefficients described by equations (3) and (4).

$$f ( t ) = \frac{{a}_{0}}{2} + {\sum }_{n = 1}^{∞} ( {a}_{n} cos n {ω}_{o} t + {b}_{n} sin n {ω}_{o} t ) , where {ω}_{o} = \frac{2 π}{T} \qquad ( 2 )$$

and where

$$\begin{matrix}{a}_{n} = \frac{2}{T} {\int }_{- T / 2}^{T / 2} f ( t ) cos n {ω}_{o} t ⅆ t , n = 1 , 2 , 3 , … & ( 3 ) \\ {b}_{n} = \frac{2}{T} {\int }_{- T / 2}^{T / 2} f ( t ) sin n {ω}_{o} t ⅆ t , n = 1 , 2 , 3 , … & ( 4 )\end{matrix}$$

T is the period of the function, and t is time.

The pressure waveforms generated by the treatment device 40 can be approximated by a Fourier series using equations (2) through (4) to facilitate research in determining the effectiveness of various types of pressure waveforms used in the therapeutic treatment of the symptoms of Meniere's disease as well as treating the symptoms, tinnitus, vertigo, fullness and intermittent loss of hearing, individually. For example, researchers can use the method of the present invention to develop different pressure waveforms for use in patients having different conditions and characteristics, such as different anthropometrical sizes. The pressure waveforms according to the present invention can include a single series of pulses or intermittent series of pulses with the time intervals between series of pulses varying as desired and the number of pulses in each series varying as desired to optimize treatment dependent upon the needs of individual patients.

As is apparent from the above, the present invention recognizes that one of the most important attributes in a pressure waveform communicated to the middle ear of a human ear to treat Meniere's disease is the first derivative which should vary and not be zero and that describing the pressure waveform as a theoretical, non-empirical, function, therapeutic benefit can be measured as a function of the variables or attributes of the pressure waveform. The pressure generator 42 for generating the pressure waveform can have any desired structure; and, in particular, the structure shown and described in U.S. Pat. No. 4,757,807 and No. 6,159,171 and in publications WO 83/0255, WO 93/08775 and WO 00/01346 can be controlled and/or modified to provide the pressure waveforms.

Having described embodiments of devices and methods for generating pressure waveforms with varying first derivatives, suitable for treating symptoms of Meniere's disease, it is believed that other modifications, variations and changes will be suggested to those skilled in the art in view of the teachings set forth herein. It is therefore to be understood that all such variations, modifications and changes are believed to fall within the scope of the present invention as defined by the appended claims. Although specific terms are employed herein, they are used in their ordinary and accustomed manner only, unless expressly defined differently herein, and not for purposes of limitation.

What is claimed is:

1. A method of treating a condition of a human ear having a middle ear and an inner ear comprising
generating a predetermined pressure wave approximated by a combination of a plurality of sinusoidal waves having different frequencies; and
delivering the pressure wave to the middle ear.

2. The method of claim 1 wherein the pressure wave has a varying first derivative.

3. The method of claim 1 wherein the pressure wave is a square wave.

4. The method of claim 1 wherein the pressure wave is a saw tooth wave.

5. The method of claim 1 wherein the pressure wave is approximated by a Fourier series.

6. The method of claim 1 wherein said method is a therapeutic treatment of Meniere's disease.

7. The method of claim 1 wherein said method is a therapeutic treatment of tinnitus.

8. The method of claim 1 wherein said method is a therapeutic treatment of vertigo.

9. The method of claim 1 wherein said method is a therapeutic treatment for hearing loss.

10. The method of claim 1 wherein said method is a therapeutic treatment for fullness.

11. The method of claim 1 wherein the predetermined pressure wave is approximated by
$$f ( t ) = \frac{{a}_{0}}{2} + {\sum }_{n = 1}^{∞} ( an cos n {ω}_{o} t + bn sin n {ω}_{o} t ) , where {ω}_{o} = \frac{2 π}{T}$$
and where
$$\begin{matrix}{a}_{n} = \frac{2}{T} {\int }_{- T / 2}^{T / 2} f ( t ) cos n {ω}_{o} t ⅆ t = 1 , 2 , 3 , … , and \\ {b}_{n} = \frac{2}{T} {\int }_{- T / 2}^{T / 2} f ( t ) sin n {ω}_{o} t ⅆ t , n = 1 , 2 , 3 , …\end{matrix}$$
and where T is a period of f(t) and t is time.

12. An apparatus for treating symptoms of a disease of the human ear having a middle ear and an inner ear comprising
a pressure generator configured to generate a pneumatic pressure wave for delivery to the middle ear to affect fluid distribution in the inner ear; and
a control unit coupled with said pressure generator and configured to output control signals to control said pressure generator to generate said pneumatic pressure wave in a pattern that can be approximated by a plurality of sine waves.

13. The apparatus of claim 12 wherein said apparatus produces a pressure wave having a first derivative effective for treating Meniere's disease.

14. The apparatus of claim 13 wherein said pneumatic pressure wave is a square wave.

15. The apparatus of claim 13 wherein said pneumatic pressure wave is a saw tooth wave.

16. The apparatus of claim 15 wherein said pneumatic pressure wave is a combination of at least two sinusoidal waves.

17. The apparatus of claim 12 wherein said pressure wave can be approximated by:
$$f ( t ) = \frac{{a}_{0}}{2} + {\sum }_{n = 1}^{∞} ( {a}_{n} cos n {ω}_{o} t + {b}_{n} sin n {ω}_{o} t ) , where {ω}_{o} = \frac{2 π}{T}$$
and where
$${a}_{n} = \frac{2}{T} {\int }_{- T / 2}^{T / 2} f ( t ) cos n {ω}_{o} t ⅆ t = 1 , 2 , 3 , … , \\ {b}_{n} = \frac{2}{T} {\int }_{- T / 2}^{T / 2} f ( t ) sin n {ω}_{o} t ⅆ t , n = 1 , 2 , 3 , …$$
and where T is a period of f(t) and t is time.

18. A method of treating a human ear having a middle ear and an inner ear separated by a partition to treat the symptoms of Meniere's disease comprising the steps of
generating a pressure pulse waveform with the pulses having a first derivative not zero and variable; and
communicating the pressure pulse waveform to the middle ear to displace the partition and affect fluid distribution in the inner ear.

19. A method of treating a human ear having a middle ear and an inner ear separated by a partition to treat the symptoms of Meniere's disease comprising the steps of
generating a pressure waveform other than a single frequency sine wave; and
communicating the pressure waveform to the middle ear to displace the partition and affect fluid distribution in the inner ear.
